(19) United States
(12) Patent Application Publication
(10) Pub. No.: US 2025/0311720 A1
(43) Pub. Date: Oct. 9, 2025

(54) POLYMERIC GEL-BASED AGROBIOLOGICAL COMPOSITION

(71) Applicant: FIB-SOL Life Technologies Pvt. Ltd., Taramani, Chennai (IN)

(72) Inventors: Kavitha SAIRAM, Perungudi Chennai (IN); Pramal BISWA, Perungudi Chennai (IN); Bijja RAJASHEKHAR, Perungudi Chennai (IN)

(21) Appl. No.: 18/865,189

(22) PCT Filed: Apr. 7, 2023
(86) PCT No.: PCT/IB2023/053573

§ 371 Date: Nov. 12, 2024

(30) Foreign Application Priority Data
May 13, 2022 (IN) 202241027681

(51) Int. Cl. A01N 25/04; A01N 63/20; A01N 63/22; A01N 63/23; A01N 63/27; A01P 15/00

(52) U.S. Cl. CPC A01N 25/04; A01N 63/20; A01N 63/22; A01N 63/23; A01N 63/27; A01P 15/00

(57) ABSTRACT

The present invention discloses a polymeric gel-based agrobiological compositions and a process for preparation thereof. Moreover, the said compositions comprise a carrier loaded with microbiological entity/s at a density higher than 1×10¹² cfu/mL and a shelf life of at least 12 months. The compositions of the present invention are stable, lightweight, condensed and highly water-dispersible that may be used as a biofertilizer, biocontrol agent, or a combination thereof leading to enhanced crop yield, plant growth, root growth, seed germination, crop quality, soil fertility. Further, the said compositions have applications in seed coating, chemical-nutrient coating, soil application, hydroponic application, foliar spray.

### CROSS-REFERENCE TO RELATED APPLICATIONS AND PRIORITY

The present application claims priority from Indian patent application Ser. No. 20/224,1027681 filed on 13 May 2022, the entirety of which is incorporated herein by a reference.

### TECHNICAL FIELD

The present invention is directed to the field of an agrobiological composition. The described subject matter, in general, relates to a polymeric gel-based agrobiological composition and the process of preparing the same. Further, the composition comprises a high payload of microbiological entities and a long shelf life.

### BACKGROUND

The Green Revolution transformed agricultural practices, bringing forth innovations and mechanization aimed towards obtaining higher yields by using newer robust varieties of crops and fertilizers; the spread however affecting agricultural sustainability. While the use of chemical fertilizers raised productivity, the benefits remained short-lived. Drawbacks such as depleting soil quality have challenged the use of chemical fertilizers and provided impetus for developing natural means like microorganisms which have transpired as a reliable alternative for providing productive and sustainable solutions for addressing increased demands on the global food chain.

Nutrients are essential for plant growth and metabolism. In order to replenish the soil microorganisms are an excellent alternative for fixing, solubilizing, mobilizing, and recycling nutrients. Even though microorganisms are natural inhabitants of soil, their population is often inadequate for serving the above functions.

Plant nutrients like nitrogen (N), phosphorus (P) and potassium (K) are highly essential for plant growth and metabolism and these macro and other micronutrients are continually absorbed from the soil in large quantities. For steady soil replenishment, particularly in an agricultural ecosystem, microorganisms offer an excellent alternative technology for fixing, solubilizing, mobilizing, and recycling nutrients. While microorganisms are natural inhabitants of soil, their population is often inadequate for serving the above functions.

A composite of such microbial inoculants of one or several types and strains of microorganisms may be packaged in a suitable carrier that provides a safe environment and shelters them from harsh conditions. Microbial consortia like these are attractive as bio-fertilizers, if upon storage, they are easy to revive and establish in the soil. Moreover, biofertilizers, being primarily soil inhabitants and part of the existing ecosystem, do not disturb soil chemical balance, like acid-base, nor skew the soil composition.

Biofertilizer requirements for a particular crop are specific and so are their combinations of N-fixing, P-solubilizing, K-mobilizing bacteria, iron-solubilizing, zinc-solubility, sulfur-solubilizing. For instance, use of non-specific Rhizobium as fertilizer, does not lead to root nodulation and consequently lead to the desired increase in crop production. Further, other considerations that impact the efficiency of microbial fertilizers are soil characteristics, such as moisture content, pH, temperature, organic matter, and other resident micro-organisms; with other factors being unfavourable, microbial fertilizers may not effectively enhance soil fertility.

Fungi, bacteria, viruses, and nematodes are examples of disease-causing organisms in plants. These organisms cause bacterial wilt, crown gall, aster yellows, basal rot, powdery mildews, and other plant diseases. Plant pests includes aphids, whiteflies, cutworms, mealybugs, leaf miners, and other insects. It is necessary to prevent and reduce such illnesses and their producing factors in order to obtain a healthy yield of plant products.

Traditionally, chemical pesticides have been used to prevent and minimise infections and disease-causing agents. However, they pose significant risk in that if not used properly, they may harm human health and environment. Further, their extensive use may lead to resistance in plants. As a result, alternative products with diverse modes of action are needed to aid resistance control. Biological control agents that are able to compete in such field conditions, are ecological, and limit the growth of illness-causing microorganisms as well as impart antibiotic effects on undesired microorganisms are needed.

To increase nutrient availability to agricultural plants, promote plant development, control or suppress plant disease, and improve soil structure, a variety of microorganisms (bacteria and fungi) have been used in soil inoculations. Further, such microorganisms may also mineralize organic pollutants or waste. However, all the above applications are currently being pursued separately, increasing treatment time and cost. As a result, an agrobiological composition must be prepared that may conduct all the activities in a combined formulation that meets the requirements.

Furthermore, packaging of biofertilizers for storage (bottles, bags, or boxes) and transport and their ease of application at the point of use like fields, farms, of any scale, size or dimension requires consideration. Thus, packets holding the entire content for use at a single lot may be difficult for small farms, also the packaging may be permeable to the entry of moisture and gases and the package once opened may get exposed to sunlight increasing chances of product deterioration.

Currently, the agrobiological compositions available in market are having certain drawbacks such as low microbial payload of about 10⁴ to 10⁹ and low shelf-life of 3-6 months. These drawbacks further causes problems such as requiring a sizable amount of agrobiological composition to be used in crop fields which increases the amount required for every yield.

### SUMMARY

The present invention discloses a gel-based agrobiological composition comprising a carrier comprising at least one water-soluble and/or biodegradable polymer and at least one microbiological entity; wherein the said carrier is loaded with the microbiological entity/s at a density higher than 1×10¹² cfu/mL wherein the said composition has a shelf life of at least 12 months.

Another embodiment of the present invention is directed toward a process for the preparation of a gel-based agrobiological composition, the said process comprising the steps of preparing a solution of a carrier comprising at least one water-soluble and/or biodegradable polymer; mixing the solution with a concentrate of one or more microbiological entity/s at a density higher than 1×10¹² cfu/mL to obtain a homogenous gel-based composition wherein the said composition has a shelf life of at least 12 months.

In another embodiment, the present invention discloses a gel-based agrobiological composition comprising a carrier comprising at least one of PVA, Xanthan gum, starch, or a combination thereof; and at least one microbiological entity selected from Azospirillum brasilense, A lipoferum, Azotobacter chroococcum, Azotobacter vinelandii, Rhizobium leguminosarum, Pseudomonas fluorescens, Bacillus megaterium, Bacillus polymyxa, Frateuria aurantia, Bacillus mucilaginous, Thiobacillus ferrooxidans, Bacillus aryabhattai, B. thuringiensis, Gluconacetobacter diazotrophicus Pseudomonas striata, P. fluorescence, Serratia liquefaciens, S. marcescens, Thiobacillus thioparus, T. neapolitanus, T. denitrificans, T. thiooxidans T. ferrooxidans, T. acidophilus, Paracoccus denitrificans, P. versutus, Xanthobacter tagetidis, Aspergillus, Fusarium, Penicillium, Piriformospora, Phoma, Funneliformis, Rhizophagus, Glomus, Trichoderma harzianum, T. viride, Metarhizium anisoplae, M. album, M. flavoviride, Beauveria bassiana, Verticillium lecanii, Paecilomyces lilacinus, Ampelomyces quisqualis, Hirsutella thompsonii or combinations thereof; wherein the said carrier is loaded with the microbiological entity/s at a density higher than 1×10¹² cfu/mL; wherein the said composition has a shelf life of at least 12 months.

This summary is not intended to identify all the essential features of the claimed subject matter, nor is it intended to use in determining or limiting the scope of the claimed subject matter.

### BRIEF DESCRIPTION OF DRAWINGS

The detailed description of the drawings is outlined with reference to the accompanying figures. In the figures, the left-most digit(s) of a reference number identifies the Figure in which the reference number first appears. The same numbers are used throughout the drawings to refer like features and components.

FIG. 1 depicts product image of the FIB-SOL NPK COMBO, a representative product from polymer gel biofertilizers

FIG. 2 demonstrates the effect of application of FIBSOL Gel and commercial biofertilizers along with other treatments on growth and yield of Maize

### DETAILED DESCRIPTION

Before the present composition and method are described, it is to be understood that this disclosure is not limited to a particular composition and method as described, as there can be multiple possible embodiments that are not expressly illustrated in the present disclosure but may still be practicable within the scope of the present disclosure.

An embodiment of this invention comprises a biodegradable, stable, lightweight, condensed and highly water-dispersible polymer composition for use as an agrobiological composition.

In another embodiment, a composition with a long shelf life and high payload or CFU count that further increases crop production, quality, and yield has been disclosed. In a related embodiment, the composition requires reduced storage facility and transportation efforts.

One embodiment of this invention comprises a partial replacement/substitute for the toxic and expensive agrobiological products used and to restore soil health.

In yet another embodiment of the present disclosure a polymer gel having immediate use in diverse agriculture applications like seed coating, chemical-nutrient coating, soil application, hydroponic application, foliar spray, and/or in combination thereof has been described.

In a further embodiment, a suitable carrier material composition having good moisture absorption capacity (high water-holding capacity), good pH buffering capacity and easily adjustable pH that is uniformly stabile, free of lump-forming tendency, compatible with many bacterial or fungal species and strains has been disclosed. Further, the composition of the present invention is suitable for storage and inoculation, is biodegradable, economical and environmentally sustainable.

In an exemplary embodiment the present invention, a composition having a unique, stable, lightweight, condensed and highly water-dispersible polymer having a long shelf life and high payload or high carrier capacity which is further used as a replacement to toxic and expensive agrobiological used having several agricultural applications as mentioned in earlier embodiment.

### DEFINITIONS

For better representation and understanding of the present invention, several terms have been defined.

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” “in an embodiment” or “in a related embodiment” in places throughout the specification are not necessarily all referring to the same embodiment. Furthermore, the features, structures or characteristics may be combined in any suitable manner in one or more embodiments.

The term “composition”, or “compositions” refers to a specific formulation of active/functional elements or components/constituents/ingredients in a mixture or as a whole with essential and compatible components and carriers for the intended use or a product comprising the specified ingredients in the specified amounts or any product which results, directly or indirectly, from a combination of the specified ingredients in the specified amount.

The term “polymer” or “polymeric” refers to any of a class of natural or synthetic substances composed of very large molecules, called macromolecules, which are multiples of simpler chemical units called monomers.

The term “microbiological entity” or “microbe” or “microbial” refers to any microscopic organisms that exist as unicellular, multicellular, or cell clusters such as bacteria or fungi, which are beneficial to agricultural practices but not limited to, bacteria or fungi.

The term “carrier” refers to a material used to transport/deliver/hold any other material living or non-living. Generally used to deliver material at a specific area or a specific condition but not limited to these.

The term “CFU” or “colony-forming unit” refers to a unit of measurement of microbiological entities. CFU estimates the number of bacteria or fungal cells in a viable sample but is not limited to bacterial or fungal cells.

### DESCRIPTION

The technical solutions offered by the present disclosure are clearly and completely described below. Examples in which specific compounds or conditions may not have been specified have been conducted under conventional conditions or in a manner recommended by the manufacturer.

The present invention intends to disclose a polymeric gel-based agrobiological composition comprising a carrier comprising at least one water-soluble and/or biodegradable polymer and at least one microbiological entity; wherein the said carrier is loaded with the microbiological entity/s at a density higher than 1×10¹² cfu/mL and the said composition has a shelf life of at least 12 months.

In a related embodiment, the said composition further comprises osmoregulators selected from inorganic ions, salts, proteins, or combinations thereof. Osmotic shock (dehydration, rehydration) is caused by fast changes in environmental factors such as temperature, pH, radiation, and osmotic pressure, which may lead to cell death. Osmoregulatory activities are meant to maintain cell turgor, allowing bacteria to proliferate under optimal conditions. Osmoregulators are soluble organic compounds that have a favorable effect on membrane stability and protein synthesis while not interfering with cellular activities. Glycerol, inorganic ions, salts, proteins, and other osmoregulators are examples. HSP16 or chaperons are examples of proteins that may act as osmoregulators.

The present invention further discloses the gel-based agrobiological composition comprises an effective amount of carrier materials. The said carrier material comprises at least one water-soluble and/or biodegradable polymer. The said carrier is a polymeric material that encapsulates a microbiological entity/s intending to protect it/them from outside conditions like pathogenic microbes, moisture, temperature, or other severe problems of the environment. The said composition has a shelf life of at least 12 months. Further, the invention discloses the loading capacity of the microbiological entity/s of higher than 1×10¹² cfu/mL. Thus, a high payload of bacteria is released into the soil even by using a small volume of the product further reducing the volume required per batch which further increases the crop production and quality.

The present invention discloses the polymer composition having consistency of liquid, sol, sol-gel, or gel. More preferably, the polymer composition is gel-based. Gel-based composition has advantages such as being easy to use, simple application procedure on any surface, and being washable. Gel composition requires less volume than any liquid composition. Such compositions have easy spreadability and higher stability for a longer time than liquid compositions.

In one embodiment of the present invention, the water-soluble and/or biodegradable polymer, comprises but is not limited to, polyvinyl alcohol (PVA), poly (ethylene glycol) (PEG), polyvinyl pyrrolidine (PVP), polyacrylic acid (PAA), polyacrylamides, poly (methacrylic acid), poly (hydroxyalkyl acrylates), poly (styrene sulfonic acid), polyesters, polyamides, polyacetals, polyethers, poly (alkylene oxides), polycarbonates, polycarboxylates, N-(2-Hydroxypropyl) methacrylamide (HPMA), divinyl ether-malcic anhydride (DIVEMA), polyphosphates, polyphosphazene, starch, xanthan gum, pectin, lignin, chitin, chitosan, dextran, carrageenan, guar gum, hydroxypropyl methylcellulose (HPMC), hydroxypropyl cellulose (HPC), hydroxyethylcellulose (HEC), sodium carboxymethylcellulose (Na-CMC), hyaluronic acid, albumin, starch, locust bean gum, gellan gum, psyllium gum, okra gum, acacia gum, karaya gum, ghatti gum, tragacanth gum or a combination thercof.

In an exemplary embodiment, at least one water-soluble and/or biodegradable polymer is polyvinyl alcohol (PVA), xanthan gum, starch, or combinations thereof.

In accordance with an exemplary embodiment of the present disclosure, a gel-based agrobiological composition comprising a carrier at least one of PVA, xanthan gum, starch, or combination thereof.

The said carrier comprises PVA, xanthan gum and starch in a ratio of 10.0:0.00:0.00 to 10.0:3.0:3.0.

In a related embodiment of the present invention, the carrier loaded with microbiological entity/s is stable at temperatures ranging from 1°° C. to 60° C.

The said carrier comprises at least one of water-soluble and/or biodegradable polymer has a concentration ranging from 10-95%. More preferably, the water-soluble and/or biodegradable polymer has a concentration ranging from 55-95%.

The said concentration of water-soluble and/or biodegradable polymer are mixed in a specific ratio such that they help in delivering consortia of microbiological entities in agriculture in a unique and stable composition with enhanced quality, longer shelf life, least/zero contamination, immediate use, handling comfort, and least logistic cost.

The carrier material composition is selected such that it provides a suitable microenvironment for the target microorganism. Additionally, it should have suitable physical and chemical properties like good moisture absorption capacity (high water-holding capacity), good pH buffering capacity, and easily adjustable pH. Also, it should have uniform stability during the processing and application, be free of lump-forming tendency and be compatible for mixing with other compounds (nutrients, adjuvants) and standard machinery applications.

In various embodiments of the present invention, the said microbiological entity is one or more bacterial or fungal cells. In another embodiment, the concentration of the said microbiological entity is in the range of 1-90% (w/v); more preferably, 1-30% (w/v) of the total composition. The working range of the said microbiological entity is 1-5%.

The composition should be such that it is compatible with as many bacterial or fungal species and strains as possible and easy to handle and process.

In a related embodiment, the bacterial or fungal cells display nitrogen-fixing, phosphate solubilizing, potassium mobilizing, zinc solubilizing, sulfur solubilizing, iron solubilizing activities or combinations thereof. Further, they may show action against diseases such as Root-Knot Nematodes, Bacterial wilt (Ralstonia solanacearum and Pseudomonas solanacearum), Fruit Rot Anthracnose (Colletotrichum capsica), Tobacco mosaic virus (TMV), Squash mosaic virus (SqMV), Caterpillar (Spodoptera litura), Cucumber mosaic virus (CMV), Damping off, Leaf spot, Fungal wilt (Fusarium), Whitefly (Bemisia tabaci), Thrips (Thrips tabaci), Grubs (White grubs: Holotrichia), Mealybug (Ferrisia virgata), Red spider mite (Tetranychus).

The said bacterial cells are selected from but are not limited to Azospirillum brasilense, A lipoferum, Azotobacter chroococcum, Azotobacter vinelandii, Rhizobium leguminosarum, Pseudomonas fluorescens, Bacillus megaterium, Bacillus polymyxa, B. subtilis, Frateuria aurantia, Bacillus mucilaginous, Thiobacillus ferrooxidans, Bacillus aryabhattai, B. thuringiensis, Gluconacetobacter diazotrophicus Pseudomonas striata, P. fluorescence, P. putida, Serratia liquefaciens, S. marcescens, Thiobacillus thioparus, T. neapolitanus, T. denitrificans, T. thiooxidans T. ferrooxidans, T. acidophilus, Paracoccus denitrificans, P. versutus, Xanthobacter tagetidis or any combinations thereof.

The said fungal cells are selected from but are not limited to Aspergillus, Fusarium, Penicillium, Piriformospora, Phoma, Funneliformis, Rhizophagus, Glomus, Trichoderma harzianum, T. viride, Metarhizium anisoplae, M. album, M. flavoviride, Beauveria bassiana, Verticillium lecanii, Paecilomyces lilacinus, Ampelomyces quisqualis, Hirsutella thompsonii or any combinations thereof.

In another related embodiment, the said composition is used as a biofertilizer, biocontrol agent or combinations thereof. The biofertilizers and biocontrol agents are used for seed coating, chemical-nutrient coating, soil application, hydroponic application, foliar spray, and/or combinations thereof.

The use of the biofertilizers and biocontrol agents lead to enhanced crop yield, plant growth, root growth, seed germination, crop quality, soil fertility. The biocontrol agents are used to prevent plants from disease causing organisms such as bacteria, fungi, viruses, nematodes. The biocontrol agents have a mode of action such as antibiotic and lytic enzyme production or lectin-based mycoparasitism, which is further involved in prolonged plant immunity (induced plant resistance), rupture of host cells caused by cry toxins, and rupture of target organism's internal organs caused by fungal toxins or fungal hyphac.

The said composition is lightweight, condensed, has high water solubility and adhesive property.

Moreover, said compositions pose no harm to the environment by being biodegradable, free of toxic materials, non-polluting, and with no environmental risks (dispersal of cells to the atmosphere or groundwater). Furthermore, the compositions are economically and environmentally sustainable.

Additionally, the composition should be suitable for storage and inoculation having a sufficient shelf life, good adherence, and allowing a rapid or controlled release of the microorganisms into the soil.

In another embodiment of the present invention, a process for the preparation of a gel-based agrobiological composition, the said process comprising the steps of; preparing a solution of a carrier comprising at least one water-soluble and/or biodegradable polymer mixing the solution with a concentrate of one or more microbiological entity/s at a density higher than 1×10¹² cfu/mL; to obtain a homogenous gel-based composition; wherein the said composition has a shelf life of at least 12 months.

In another embodiment of the present invention, a process wherein the bacterial or fungal cells are selected from the group as mentioned in the earlier embodiment.

In addition, the carrier is prepared from the selected water-soluble and/or biodegradable polymer by mixing in a particular amount such that the carrier may maintain the water activity of microbes thereby supporting their health and stability. Further, the carrier is prepared at a temperature ranging from 50-120° C.; preferably, 70-100° C. and at a pH ranging from 5.5-7.5.

In another embodiment of the present invention discloses the concentrate of microbiological entity/s and carrier solution are mixed in a ratio ranging from 1:5 to 5:1; more preferably, 1:1 to 1:4. The solution is then gently blended under sterile conditions using an automated stirring machine till a homogeneous solution is obtained. The final solution is then automatically packed into 16 mm spout mouth aluminium pouches and stored under refrigerated condition.

The process for the preparation of the polymer composition is cost-effective and economic. The materials used are non-harmful, eco-friendly, and biodegradable. The process of preparing the polymer composition is less laborious.

An exemplary embodiment of the present invention discloses a gel-based agrobiological composition comprising: a carrier comprising at least one of PVA, Xanthan gum, starch, or a combination thereof; and at least one microbiological entity selected from Azospirillum brasilense, A lipoferum, Azotobacter chroococcum, Azotobacter vinelandii, Rhizobium leguminosarum, Pseudomonas fluorescens, Bacillus megaterium, Bacillus polymyxa, Frateuria aurantia, Bacillus mucilaginous, Thiobacillus ferrooxidans, Bacillus aryabhattai, B. thuringiensis, Gluconacetobacter diazotrophicus Pseudomonas striata, P. fluorescence, Serratia liquefaciens, S. marcescens, Thiobacillus thioparus, T. neapolitanus, T. denitrificans, T. thiooxidans T. ferrooxidans, T. acidophilus, Paracoccus denitrificans, P. versutus, Xanthobacter tagetidis, Aspergillus, Fusarium, Penicillium, Piriformospora, Phoma, Funneliformis, Rhizophagus, Glomus, Trichoderma harzianum, T. viride, Metarhizium anisoplae, M. album, M. flavoviride, Beauveria bassiana, Verticillium lecanii, Paecilomyces lilacinus, Ampelomyces quisqualis, Hirsutella thompsonii or combinations thereof, wherein the said carrier is loaded with the microbiological entity/s at a density higher than 1×10¹² cfu/mL; wherein the said composition has a shelf life of at least 12 months.

### EXAMPLES

The term “gel-based polymer composition” or “lightweight polymer composition” or “polymer gel composition” or “gel-based composition” all refers to the gel-based agrobiological composition and may be used alternatively throughout the specification.

### Example 1

### Process of Preparation of Polymer Gel Biofertilizer

10.2 g of polyvinyl alcohol (PVA), 0.05 g of starch, and 0.04 g of xanthan gum are added with 85 ml DM water at 90° C. until a homogeneous polymer solution was achieved. This was further stored at 6° C., until use.

Culture media was prepared in a conical flask and sterilized at 121° C. (15 psi) for 20 mins which was further cooled to RT and inoculated with a loopful of corresponding bacterial culture (see Table 1). Inoculated media was kept at 27-37° C. in a shaker incubator for 24-72 hours. 100 L of media was inoculated with 10 L inoculum.

The broth was passed through the centrifuge at the flow rate of 10 L/hr. After the run, the bacterial cake was aseptically taken out and the pelleted microbiological cake was aseptically collected. Pellet (0.1 g) was reconstituted in osmo-regulator (10 ml) buffer to make cell solution which is henceforth known as concentrate. Quality analysis using the method of standard plate count is performed to estimate culture purity and microbiological load.

Concentrate and polymer gel was brought to room temperature and mixed in a ratio of 1:4 respectively. The solution was then gently blended under sterile conditions using an automated stirring machine till a homogeneous solution was obtained. The final solution was then automatically packed into 16 mm spout mouth aluminium pouches and stored under refrigerated condition.

For NPK Combo products, the requisite quantity of prepared gel of different microbe was mixed and re-blended.

TABLE 1
Bacterial and fungal strains for inoculation

| Microbes used | Genus | Strain |
|---|---|---|
| Nitrogen- | Azospirillum | Azospirillum brasilense, |
| fixing bacteria | Azotobacter | A lipoferum |
| | Rhizobium | Azotobacter chroococcum, |
| | Nitrogen fixation | Azotobacter vinelandii |
| | | Rhizobium leguminosarum |
| Phosphate | Pseudomonas | Pseudomonas fluorescens |
| solubilizing | Bacillus | Bacillus megaterium |
| bacteria | | Bacillus polymyxa |
| Potassium | Frateuria | Frateuria aurantia |
| mobilizing | Bacillus | Bacillus mucilaginous |
| bacteria | | |
| Iron | Thiobacillus | Thiobacillus ferrooxidans |
| solubilizing | | |
| bacteria | | |
| Zinc | Ppseudomonas | Bacillus aryabhattai, |
| solubilizing | Bacillus | B. thuringiensis, |
| bacteria | Gluconacetobacter | Gluconacetobacter |
| | Serratia | diazotrophicus |
| | | Pseudomonas striata, |
| | | P. fluorescence, |
| | | Serratia liquefaciens, |
| | | S. marcescens, |
| Sulphur | Thiobacillus | Thiobacillus thioparus, |
| oxidizing | Paracoccus | T. neapolitanus, |
| bacteria | Xanthobacter | T. denitrificans, |
| | | T. thiooxidans |
| | | T. ferrooxidans, |
| | | T. acidophilus, |
| | | Paracoccus denitrificans, |
| | | P. versutus, |
| | | Xanthobacter tagetidis. |
| Agriculturally | Trichoderma, | Trichoderma harzianum, |
| important | Metarhizium, | T. viride, Metarhizium |
| fungi | Beauveria, | anisoplae, M. album, |
| | Verticillium | M. flavoviride, Beauveria |
| | Paecilomyces | bassiana, Verticillium |
| | Ampelomyces | lecanii, Paecilomyces |
| | Hirsutella | lilacinus, Ampelomyces |
| | | quisqualis, Hirsutella |
| | | thompsonii |

### Example 2

### Studies Showing the Different Compositions of Polymer Gel Biofertilizer

Different compositions (see Table 2) of PVA, xanthan gum and starch were studied to determine the composition showing the best results (cfu/mL).

The viability of Azospirillum in different carrier compositions was studied under three different storage temperatures (6° C., 25° C. & 55° C.) and the results are analyzed in Table 2.

Composition 6 (11% (PVA-Starch-Xanthan Gum)+Azospirillum) was found to be superior to the others in the study, cell viability, and formulation stability were consistent across all the storage conditions.

TABLE 2
Cell viability in different compositions after 24 hrs of incubation

| | | Storage | Stability After 24 hrs (CFU/ml) | | | | | |
|---|---|---|---|---|---|---|---|---|
| Compositions | | Temp. | 10⁰ | 10⁻⁴ | 10⁻⁶ | 10⁻⁸ | 10⁻¹⁰ | Remarks |
| 1 | 8% PVA + | @6° C. | TNTC | TNTC | 30 | NIL | NIL | Microbial stability issues |
| | Azospirillum | @25° C. | TNTC | TNTC | 4 | NIL | NIL | Microbial stability issues. Bulging |
| | | | | | | | | of the pack |
| | | @55° C. | TNTC | TNTC | 109 | 75 | 55 | Microbial stability issues. Bulging |
| | | | | | | | | of the storage pack |
| 2 | 10% PVA + | @6° C. | TNTC | TNTC | 9 | NIL | NIL | Microbial stability issues |
| | Azospirillum | @25° C. | TNTC | TNTC | 4 | 2 | 4 | Microbial stability issues. Bulging |
| | | | | | | | | of the storage pack |
| | | @55° C. | TNTC | TNTC | 180 | 125 | 100 | Microbial stability issues. Bulging |
| | | | | | | | | of the storage pack |
| 3 | 0.8% Xanthan | @6° C. | TNTC | TNTC | TNTC | TNTC | TNTC | |
| | Gum + | @25° C. | TNTC | TNTC | TNTC | TNTC | TNTC | Carrier Consistency is lost. Bulging |
| | Azospirillum | | | | | | | of the storage pack |
| | | @55° C. | TNTC | NIL | NIL | NIL | NIL | Microbial stability issues. Carrier |
| | | | | | | | | consistency is lost. Bulging of the |
| | | | | | | | | storage pack |
| 4 | 5% Starch + | @6° C. | TNTC | TNTC | TNTC | 148 | 40 | Microbial stability issues. |
| | Azospirillum | @25° C. | TNTC | TNTC | 120 | 54 | NIL | Microbial stability issues. Bulging |
| | | | | | | | | of the pack |
| | | @55° C. | TNTC | NIL | NII | NIL | NIL | Microbial stability issues. Bulging |
| | | | | | | | | of the storage pack |
| 5 | 11% (PVA- | @6° C. | TNTC | TNTC | TNTC | TNTC | TNTC | |
| | Starch) * | @25° C. | TNTC | TNTC | TNTC | TNTC | TNTC | Bulging of the storage pack |
| | + | @55° C. | TNTC | TNTC | TNTC | TNTC | TNTC | Bulging of the storage pack |
| | Azospirillum | | | | | | | |
| 6 | 11% (PVA- | @6° C. | TNTC | TNTC | TNTC | TNTC | TNTC | Highly stable, and with no bulging |
| | Starch-Xanthan | | | | | | | issues |
| | Gum) ** + | @25° C. | TNTC | TNTC | TNTC | TNTC | TNTC | Highly stable, and with no bulging |
| | Azospirillum | | | | | | | issues |
| | | @55° C. | TNTC | TNTC | TNTC | TNTC | TNTC | Highly stable, and with no bulging |
| | | | | | | | | issues |

*PVA: Starch = 70:30;
**PVA: Starch: Xanthan Gum = 10:0.05:0.04;
TNTC = Too Numerous To Count

### Example 3

### Studies Showing a Comparison Between Different Kinds of Polymers as a Carrier

The viability of Azospirillum in different commercial carriers like talc and lignite was compared with the viability of Azospirillum in polymer gel (N-GEL) (Table 3). Polymer gel (N-GEL) carrier was found to be highly stable and compatible with the Azospirillum over a varied storage time and at an elevated temperature of 34° C.

TABLE 3
Cell Viability Vs. Carrier for Biofertilizers (Storage temp. at 34° C.)

| | Cell Viability (CFU/mL) | | | | | |
|---|---|---|---|---|---|---|
| Carrier | Day 1 | Day 5 | Day 10 | Day 16 | Day 23 | Day 30 |
| Talc | 4.4 × 10¹¹ | 3.2 × 10¹⁰ | 9 × 10⁹ | 7.4 × 10⁸ | 3.1 × 10⁶ | 2.1 × 10⁶ |
| Lignite | 9.5 × 10⁹ | 3 × 10⁷ | 8.1 × 10⁶ | 2.9 × 10⁴ | 10 | 0 |
| Polymer | >3 × 10¹⁵ | >3 × 10¹⁵ | >3 × 10¹⁵ | >3 × 10¹⁵ | >3 × 10¹⁵ | >3 × 10¹⁵ |
| Gel (N-GEL) | | | | | | |

### Example 4

### Studies Showing a Comparison Between Commercially Available Biofertilizers, PBS and FIB-SOL N-Gel

The viability of Azospirillum was compared in different liquid carriers under a storage temperature of 34° C., over a storage period of 10 days and the results were recorded in Table 4. Polymer gel (N-GEL) was found to be superior to the other liquid carriers in the study with better cell viability (>3×10¹⁵) on day 10.

TABLE 4
Cell Viability Comparison (PBS vs Commercial Liquid
Biofertilizers vs. FIB-SOL N-Gel; Storage temp. at 34° C.)

| | Cell Viability (CFU/ml) | | |
|---|---|---|---|
| Carrier | Day 0 | Day 5 | Day 10 |
| Phosphate-Buffered Saline | 5 × 10¹⁰ | 8 × 10¹⁰ | 4 × 10¹⁰ |
| (PBS) | | | |
| Commercial Liquid | 1.5 × 10⁶ | 5.3 × 10⁶ | 2 × 10⁵ |
| Biofertilizer | | | |
| Polymer Gel (N-GEL) | >3 × 10¹⁵ | >3 × 10¹⁵ | >3 × 10¹⁵ |

### Example 5

### Studies to Support Longer Shelf Life (12 Months) and Higher CFU Count

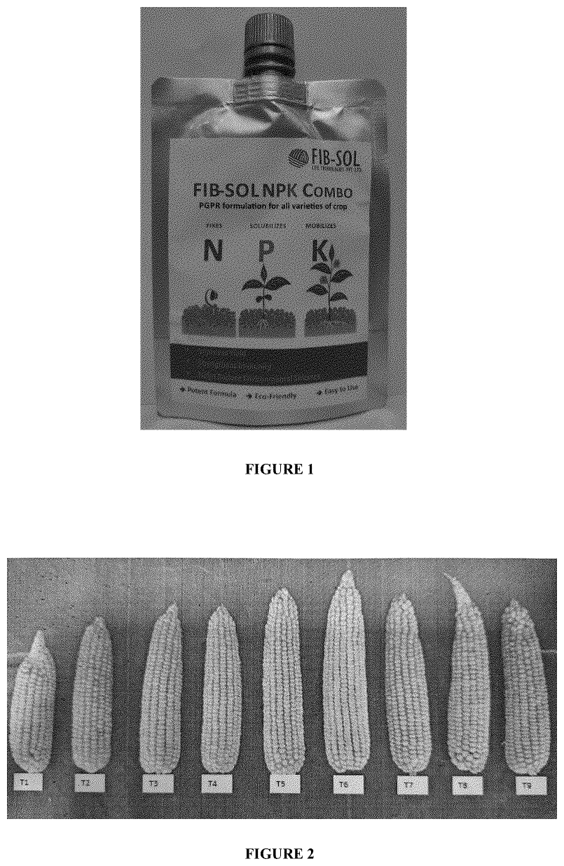

The exemplary polymer gel biofertilizer products viz. N-GEL, PK-GEL, and NPK-GEL (FIG. 1) were subjected to stability analysis. 3 sets each of 100 ml packed products (N-GEL, PK-GEL, and NPK-GEL) were placed at an accelerated temperature condition of 35° C. and were harvested every 0^{th}, 1^{st}, 3^{rd}, 6^{th}, and 12^{th} months. The samples were assessed for purity and growth using the standard plate count method. The results for stability tests are provided in table 5 whereby TNTC (Too Numerous to count) represents CFU (Colony Forming Units) count at >300.

TABLE 5
Shelf life of Polymer Gel Biofertilizer Compositions

| Time of | | Microbial Cell Count (CFU/ml) | | | | | |
|---|---|---|---|---|---|---|---|
| Study | Product | 10⁰ | 10⁻³ | 10⁻⁵ | 10⁻⁸ | 10⁻¹⁰ | 10⁻¹² |
| 0^{th} Day | N-GEL | TNTC | TNTC | TNTC | TNTC | TNTC | TNTC |
| | PK-GEL | TNTC | TNTC | TNTC | TNTC | TNTC | TNTC |
| | NPK- | TNTC | TNTC | TNTC | TNTC | TNTC | TNTC |
| | GEL | | | | | | |
| 1^{st} | N-GEL | TNTC | TNTC | TNTC | TNTC | TNTC | TNTC |
| Month | PK-GEL | TNTC | TNTC | TNTC | TNTC | TNTC | TNTC |
| | NPK- | TNTC | TNTC | TNTC | TNTC | TNTC | TNTC |
| | GEL | | | | | | |
| 3^{rd} | N-GEL | TNTC | TNTC | TNTC | TNTC | TNTC | TNTC |
| Month | PK-GEL | TNTC | TNTC | TNTC | TNTC | TNTC | TNTC |
| | NPK- | TNTC | TNTC | TNTC | TNTC | TNTC | TNTC |
| | GEL | | | | | | |
| 6^{th} | N-GEL | TNTC | TNTC | TNTC | TNTC | TNTC | TNTC |
| Month | PK-GEL | TNTC | TNTC | TNTC | TNTC | TNTC | TNTC |
| | NPK- | TNTC | TNTC | TNTC | TNTC | TNTC | TNTC |
| | GEL | | | | | | |
| 12^{th} | N-GEL | TNTC | TNTC | TNTC | TNTC | TNTC | TNTC |
| Month | PK-GEL | TNTC | TNTC | TNTC | TNTC | TNTC | TNTC |
| | NPK- | TNTC | TNTC | TNTC | TNTC | TNTC | TNTC |
| | GEL | | | | | | |

### Example 6

### Field Trial on the Effect Of FIB-SOL Gel Biofertilizer Under Crop Conditions in Maize Crop

A field experiment was conducted at Agricultural Research Station, Amaravati on Maize (Zea mays) in red soil type, in an irrigated field to evaluate polymer gel biofertilizer as Agri input.

TABLE 6
Details of the Treatments
Treatment

| | |
|---|---|
| T1 | Control (No additional inputs added |
| T2 | FIBSOL GEL |
| T3 | NPK Commercial Biofertilizer |
| T4 | Chemical: POP* + FYM** |
| T5 | Chemical: POP* + FYM** + FIBSOL Gel |
| T6 | Chemical: 75% POP* + |
| | FYM** + FIBSOL Gel |
| T7 | Chemical: 50% POP* + |
| | FYM** + FIBSOL Gel |
| T8 | University Organic Practice |
| T9 | University Organic Practice + FIBSOL Gel |

*POP = Package of Practice
**FYM = Farmyard manure

The experiment was laid out in red (light) soil with a randomized block design (RBD) design consisting of 9 treatments (Treatment details given in the above table 6) run in triplicates. The experiment was taken up during Rabi-2020 as an irrigated crop. The variety used was VMH 6477 with a spacing of 60×25 cm in a plot size of 21.6 sq. m.

Date of sowing: 01.02.2020

Date of Harvest: 21.05.2020

Number of applications: 2 (Two) basal and 30 days after sowing

Dates of application: 01.02.2020 and 03.03.2020

The results obtained in this trial on “Evaluation of polymer gel biofertilizers as Agri Input in maize” are outlined in Table 7.

In this trial application of biofertilizers improved plant growth, yield, soil nutrient status, and microbial activity in the soil during maize cultivation. FIBSOL Gel performed better over other commercial bio compositions applied. Further growth and yield were improved when FIBSOL Gel was applied with recommended POP+FYM and with university organic practice over applying FIBSOL Gel alone.

TABLE 7
Evaluation studies to prove the efficacy of Polymer Gel Biofertilizer in Maize under field
conditions (FIG. 2) (Trials conducted on Maize by NGRAU Guntur 2020)

| Maize | | | | | | |
|---|---|---|---|---|---|---|
| | Plant height | Grain yield | | 60 Days After Sowing | | |
| Treatments | (cm) | (Kg/Ha) | seed weight | N(Kg/h) | P (Kg/h) | K(Kg/h) |
| Control | 175.56 | 6933 | 21.66 | 153.25 | 24.5 | 237.33 |
| FIB-SOL Gel | 189.58 | 7508 | 24.33 | 169.33 | 32.33 | 241 |
| Commercial biofertilizer | 187.75 | 7200 | 23.66 | 165.66 | 32.66 | 242.33 |
| Chemical: POP + FYM | 195.98 | 8217 | 25.33 | 178.45 | 29.66 | 255.33 |
| Chemical: | 212.75 | 8278 | 25.66 | 183.66 | 37.5 | 262.66 |
| POP + FYM + GEL | | | | | | |
| Chemical: 75% | 197.51 | 7888 | 24.33 | 192.5 | 35 | 263.5 |
| POP + FYM + GEL | | | | | | |
| Chemical: | 195.37 | 7647 | 23.66 | 195.33 | 35.33 | 259 |
| 50% POP + FYM + GEL | | | | | | |
| Organic | 188.51 | 7500 | 23 | 197.66 | 32.55 | 253.33 |
| Organic + FIB-SOL GEL | 204.82 | 8042 | 25.33 | 205.66 | 34.66 | 261.5 |
| Se(m)± | 5.21 | 225.75 | 0.95 | | | |
| CD @5% | 14.85 | 607.5 | 2.59 | | | |

### Example 7

### Field Trial Report on the Effect of FIB-SOL Gel Biofertilizer Under Crop Conditions in Groundnut

A field experiment was conducted at Research Institute on Organic Farming (RIOF) field, UAS, GKVK, Bengaluru during Kharif 2019 (under protective irrigation through a drip) on groundnut to evaluate polymer gel biofertilizer as agri input.

The experiment was laid with a Randomized Block Design (RBD) design consisting of 9 treatments (Treatment details given in the table 8) run in triplicates. The experiment was taken up during Kharif-2019 as an irrigated crop. The variety used was GKVK-5 with a spacing of 30×10 cm in a net plot size of 3.9 m×3.3 m.

Before the layout of the experiment, complete soil samples were drawn from the experimental plot by core sampler method from the upper 0-15 cm soil layer, and soil was analyzed for chemical properties viz., pH, EC, Organic carbon, Available nitrogen, phosphorous and potassium. Biological properties viz., general soil bacteria, fungi, actinomycetes, P-solubilizer, N-fixing bacteria.

TABLE 8
Treatment Details
Treatment

| | |
|---|---|
| T1 | Control (No additional inputs added) |
| T3 | FIB-SOL GEL NPK combo |
| T4 | Commercial Biofertilizer NPK combo |
| T6 | POP + FIBSOL Gel |
| T7 | GKVK Organic practice |
| T9 | GKVK Organic Practice + FIBSOL Gel |

In conclusion, use of FIBSOL GEL biofertilizer (FIBSOL NPK combo) for improving growth & yield in organic groundnut production as well as in conventional farming was demonstrated. FIBSOL GEL biofertilizer (FIBSOL NPK combo) performed better when FYM/Vermicompost/Compost on an N equivalent basis was supplemented. This was observed when only FIBSOL Gel was applied in the void of organic manures to the crop recorded 30 per cent less yield as compared to supplementation of organic manures along with FIBSOL Gel product. Plants in the FIBSOL Gel applied plots remained greener throughout the crop season compared to the plants in the control plots.

Soil Microbial load improved in organic production systems where FIBSOL GEL biofertilizers were used. Hence, the application of FIBSOL Gel biofertilizers was easy and safe, and more effective when used in combination with organic manures (See Table 9).

TABLE 9
Evaluation studies to prove the efficacy of Polymer Gel Biofertilizer in Groundnut under
field conditions

| | | No. of | No. of | Pod | Kernel | Test | | | |
|---|---|---|---|---|---|---|---|---|---|
| | | Nodules/ | Pods/ | yield | yield | weight | N | P | K |
| Groundnut | Treatments | plant | plant | kg ha⁻¹ | kg ha⁻¹ | Kernel (g) | (kg ha⁻¹) | (kg ha⁻¹) | (kg ha⁻¹) |
| Control | T₁ | 47 | 20.13 | 448 | 277 | 31.6 | 202.5 | 11.9 | 101 |
| NPK-GEL | T₃ | 102.33 | 35.6 | 1515 | 1000 | 39.7 | 239.3 | 13.6 | 138.2 |
| Commercial | T₄ | 79 | 29.2 | 1063 | 686.4 | 35 | 220.4 | 12.3 | 128 |
| bio-fertilizer | | | | | | | | | |
| NPK combo | | | | | | | | | |
| POP + NPK- | T₆ | 105.23 | 45.8 | 2580 | 1801.33 | 45 | 260.9 | 15.3 | 152.3 |
| GEL | | | | | | | | | |
| GKVK Organic | T₇ | 108 | 40.47 | 1867 | 1269.1 | 42 | 242 | 17.3 | 140.2 |
| practice | | | | | | | | | |
| GKVK Organic | T₉ | 115.7 | 44.07 | 2268 | 1587 | 45 | 243 | 18.6 | 145.3 |
| practice + NPK- | | | | | | | | | |
| GEL | | | | | | | | | |
| | SE.m± | 2.06 | 2.08 | 109 | 100.1 | 1.08 | 13.14 | 0.83 | 2.2 |
| | C.D @ 5% | 6.17 | 6.25 | 326.79 | 300.11 | 3.25 | 39.05 | NS | NS |

### Example 8

### Field Trial Report on the Effect of FIB-SOL Gel Biofertilizer Under Crop Conditions in Tomato

A field experiment was conducted at the organic farming block of the Research Institute on Organic Farming (RIOF), University of Agricultural Sciences (UAS), GKVK, Bengaluru-65 during Kharif 2019 on tomato (Lycopersicon esculentum Mill) to evaluate Polymer Gel Biofertilizer as Agri input.

The experiment was laid with a Randomized Block Design (RBD) design consisting of 6 treatments (Treatment details given in the table 10) run in triplicates. The experiment was taken up during Kharif-2019 (under protective irrigation through a drip) as an irrigated crop. The variety used NAS 501 (Namdhari Seeds) with a spacing of 90 cm×45 cm in a net plot size of 2.7 m×2.6 m.

Before the layout of the experiment, complete soil samples were drawn from the experimental plot by core sampler method from the upper 0-15 cm soil layer, and soil was analyzed for chemical properties viz., pH, EC, organic carbon, available nitrogen, phosphorous and potassium and biological properties viz., general soil bacteria, fungi, actinomycetes, P-solubilizer, N-fixing bacteria. The soil of the experimental plot was red sandy loam, grouped under the class of alfisols. Soil pH was near neutral (6.7) with an electrical conductivity of 0.22 dS m⁻¹, soil organic carbon content was medium (0.44%) and medium in available nitrogen (320 kg ha⁻¹), phosphorous (39.8 kg ha⁻¹), and potassium (225 kg ha⁻¹).

TABLE 10
Treatment details
Treatment

| | |
|---|---|
| T1 | Control (No additional inputs added) |
| T3 | FIB-SOL GEL NPK combo |
| T4 | Commercial Biofertilizer NPK combo |
| T6 | POP + FIBSOL Gel |
| T7 | GKVK Organic practice |
| T9 | GKVK Organic Practice + FIBSOL Gel |

Use of FIBSOL gel biofertilizer (FIBSOL NPK combo) demonstrated improved growth & yield in organic tomato production as well as in conventional farming. FIBSOL GEL biofertilizer (FIBSOL NPK combo) performed better when FYM/Vermicompost/Compost on an N equivalent basis was supplemented. This was observed when only FIBSOL Gel was applied to the void of organic manures to the crop recorded 30 per cent less yield as compared to supplementation of organic manures along with FIBSOL Gel product. Plants in the FIBSOL Gel applied plots remained greener and were healthy throughout the crop season compared to the plants in the control plots. Soil Microbial load improved in organic production systems where FIBSOL GEL biofertilizers were used. Hence, it was concluded that the application of FIBSOL Gel biofertilizers is very easy and safe, and more effective when used in combination with organic manures (See Table 11).

TABLE 11
Evaluation studies to prove the efficacy of Polymer Gel Biofertilizer in Tomato under field
conditions (Trials conducted on Tomatoes by GKVK Bangalore 2020)

| | | | No. of | No. of | | | | | |
|---|---|---|---|---|---|---|---|---|---|
| | | Days to | branches/ | flower | Fruit Wt. | | | | |
| | | 50% | plant at 90 | clusters/ | kg/(10 no. | Fruit yield | | | |
| Tomato | Treatments | flowering | DAT | plant | fruits) | (t/ha) | N Kg ha-1 | P Kg ha-1 | K Kg ha-1 |
| Control | T1 | 43.7 | 4.17 | 2.4 | 0.62 | 9 | 247.4 | 21.77 | 193.34 |
| FIB-SOL | T3 | 37.05 | 5.88 | 5.1 | 0.956 | 24.3 | 272.63 | 23.78 | 210.34 |
| GEL NPK | | | | | | | | | |
| combo | | | | | | | | | |
| Commercial | T4 | 40 | 5.66 | 3.8 | 0.809 | 18 | 266.2 | 22.79 | 203.4 |
| bio- | | | | | | | | | |
| fertilizer | | | | | | | | | |
| NPK | | | | | | | | | |
| combo | | | | | | | | | |
| POP + | T6 | 37.42 | 7.15 | 6.8 | 1.28 | 39.4 | 308 | 27.61 | 235 |
| FIBSOL | | | | | | | | | |
| GEL | | | | | | | | | |
| GKVK | T7 | 34.07 | 6.36 | 5.3 | 1 | 29 | 276.3 | 24.23 | 214.3 |
| Organic | | | | | | | | | |
| practice | | | | | | | | | |
| GKVK | T9 | 32.29 | 6.85 | 5.8 | 1.169 | 33.9 | 286.3 | 26.84 | 218.16 |
| Organic | | | | | | | | | |
| practice + | | | | | | | | | |
| FIBSOL | | | | | | | | | |
| GEL | | | | | | | | | |
| | SE.m+ | 1.87 | 0.34 | 0.43 | 0.02 | 1.64 | 10.32 | 1.45 | 8.6 |
| | C.D | 5.61 | 1.02 | 1.3 | 0.04 | 4.92 | NS | NS | NS |
| | @ 5% | | | | | | | | |

### Example 9

### Studies to Support the Compatibility of Polymer Gel Biofertilizer in Chemical Nutrient Coating

1. Preparation of Urea Coating using N-Gel:

15 gms of urea and 0.5 ml of N-Gel were evenly mixed and allowed to air dry for 4 hrs without disturbance. Then. 5 g of Urea coated with N-Gel was suspended in 950 μL of sterile water. The samples were assessed for purity and growth using the standard plate count method as explained below.

100 μL from 10⁰, 10⁻³, 10⁵, 10⁻⁸, 10⁻¹⁰ and 10¹² dilution samples were plated on the Burk's media plates and incubated at 34° C. for 24-36 hrs for colonies to develop and the results are shown in Table 12. Model colonies without contamination in 10-12 plates ensure cellular density at more than 101² cells/ml of the product and N-Gel compatibility with urea.

TABLE 12
Quality analysis of urea coated with N-Gel
(polymer gel biofertilizer)

| | Carried out in | | | | | | |
|---|---|---|---|---|---|---|---|
| | triplets on | | | | | | |
| Set | BURK's | Dilutions | | | | | |
| No. | Media | 10⁰ | 10⁻³ | 10⁻⁵ | 10⁻⁸ | 10⁻¹⁰ | 10⁻¹² |
| 1 | Urea Coated | TNTC | TNTC | TNTC | TNTC | TNTC | TNTC |
| | with N-GEL | | | | | | |
| 2 | Urea Coated | TNTC | TNTC | TNTC | TNTC | TNTC | TNTC |
| | with N-GEL | | | | | | |
| 3 | Urea Coated | TNTC | TNTC | TNTC | TNTC | TNTC | TNTC |
| | with N-GEL | | | | | | |

*TNTC = too numerous to count
*N-Gel = Azotobacter

2. Preparation of Urea Coating using PK-Gel:

15 gms of urea and 0.5 ml of PK-Gel were evenly mixed and allowed to air dry for 4 hrs without disturbance. 5 g Urea coated with PK-Gel was suspended in 950 μL of sterile water. The samples were assessed for purity and growth using the standard plate count method as explained below. 100 μL from 10⁰, 10⁻³, 10⁻⁵, 10⁻⁸, 10⁻¹⁰ and 10⁻¹² dilution samples were plated on the PSB media plates and incubated at 34° C. for 24-36 hrs for colonies to develop and the results are shown in Table 13.

Model colonies without contamination in 10⁻¹² plates ensure cellular density at more than 1012 cells/ml of the product and PK-Gel compatibility with urea.

TABLE 13
Quality analysis of urea coated with PK-Gel
(polymer gel biofertilizer)

| | Carried out | | | | | | |
|---|---|---|---|---|---|---|---|
| | in triplets | | | | | | |
| Set | on PSB | Dilutions | | | | | |
| No. | Media | 10⁰ | 10⁻³ | 10⁻⁵ | 10⁻⁸ | 10⁻¹⁰ | 10⁻¹² |
| 1 | Urea | TNTC | TNTC | TNTC | TNTC | TNTC | TNTC |
| | Coated with | | | | | | |
| | PK-GEL | | | | | | |
| 2 | Urea | TNTC | TNTC | TNTC | TNTC | TNTC | TNTC |
| | Coated with | | | | | | |
| | PK-GEL | | | | | | |
| 3 | Urea | TNTC | TNTC | TNTC | TNTC | TNTC | TNTC |
| | Coated with | | | | | | |
| | PK-GEL | | | | | | |

*TNTC = too numerous to count

### Example 10

### Studies to Support The Dispersibility Of Polymer Gel Biofertilizer In Water (Instant Release Of Cells POC)

1 ml of N-Gel was suspended in 1 ml of sterile water. The sample was assessed for dispersibility of N-Gel in water using the standard plate count method as explained below.

1 ml of N-Gel and 1 ml of sterile water mixture were further serial diluted in sterile water to form 10⁰, 10⁻³, 10⁻⁵, 10⁻⁸, 10⁻¹⁰ and 10⁻¹² dilute samples.

100 μL from 10⁰, 10⁻³, 10⁻⁵, 10⁻⁸, 10⁻¹⁰ and 10⁻¹² diluted samples were plated on the DOB media plates and incubated at 34° C. for 24-36 hrs for complete development of colonies. The results are compared with the standard plate count results of N-Gel as mentioned in Table 14

Model colonies without contamination in 10⁻¹² plates ensure cellular density at more than 10¹² cells/ml of the product and complete dispersibility of N-Gel in water.

Similar results were obtained with the water dispersibility experiment of PK-Gel & NPK-Gel. Results are summarized in Table 14.

TABLE 14
Dispersibility of polymer gel biofertilizer in water

| | SPC Carried | | | | | | |
|---|---|---|---|---|---|---|---|
| | out on | Microbial Cell Counts | | | | | |
| Set | BURK's & | (CFU/Unit Volume) | | | | | |
| No. | PSB Media | 10⁰ | 10⁻³ | 10⁻⁵ | 10⁻⁸ | 10⁻¹⁰ | 10⁻¹² |
| 1 | N-GEL | TNTC | TNTC | TNTC | TNTC | TNTC | TNTC |
| | (CFU/ml) | | | | | | |
| 2 | N-GEL + | TNTC | TNTC | TNTC | TNTC | TNTC | TNTC |
| | Water | | | | | | |
| | (CFU/2 ml) | | | | | | |
| 3 | PK-GEL | TNTC | TNTC | TNTC | TNTC | TNTC | TNTC |
| | (CFU/ml) | | | | | | |
| 4 | PK-GEL + | TNTC | TNTC | TNTC | TNTC | TNTC | TNTC |
| | Water | | | | | | |
| | (CFU/2 ml) | | | | | | |
| 5 | NPK-GEL | TNTC | TNTC | TNTC | TNTC | TNTC | TNTC |
| | (CFU/ml) | | | | | | |
| 6 | NPK-GEL + | TNTC | TNTC | TNTC | TNTC | TNTC | TNTC |
| | Water | | | | | | |
| | (CFU/2 ml) | | | | | | |

The embodiments, examples and alternatives of the preceding paragraphs or the description and drawings, including any of their various aspects or respective individual features, may be taken independently or in any combination. Features described in connection with one embodiment are applicable to all embodiments unless such features are incompatible.

The preferred embodiments of the present invention are described in detail above. However, it should be understood that many modifications and changes are possible using ordinary technologies in the field according to the concept of the present invention without creative work. Therefore, all technical solutions that can be obtained by those skilled in the art through logical analysis, reasoning or limited experiments based on the concept of the present invention on the basis of the prior art should fall within the protection scope determined by the claims.

1. A gel-based agrobiological composition comprising;
a. a carrier comprising at least one water-soluble and/or biodegradable polymer; and
b. at least one microbiological entity;
wherein the said carrier is loaded with the microbiological entity/s at a density higher than 1×10¹² cfu/mL; wherein the said composition has a shelf life of at least 12 months.

2. The composition as claimed in claim 1, wherein the said composition further comprises osmoregulators selected from inorganic ions, salts, proteins, or combinations thereof.

3. The claim as claimed in claim 1, wherein the water-soluble and/or biodegradable polymer is selected from polyvinyl alcohol (PVA), poly (ethylene glycol) (PEG), polyvinyl pyrrolidone (PVP), polyacrylic acid (PAA), polyacrylamides, poly (methacrylic acid), poly (hydroxyalkyl acrylates), poly (styrene sulfonic acid), polyesters, polyamides, polyacetals, polyethers, poly (alkylene oxides), polycarbonates, polycarboxylates, N-(2-Hydroxypropyl) methacrylamide (HPMA), divinyl ether-maleic anhydride (DIVEMA), polyphosphates, polyphosphazene, xanthan gum, pectin, lignin, chitin, chitosan, dextran, carrageenan, guar gum, hydroxypropyl methylcellulose (HPMC), hydroxypropyl cellulose (HPC), hydroxyethyl cellulose (HEC), sodium carboxymethylcellulose (Na-CMC), hyaluronic acid, albumin, starch, locust bean gum, gellan gum, psyllium gum, okra gum, acacia gum, karaya gum, ghatti gum, tragacanth gum or combinations thereof.

4. The claim as claimed in claim 1, wherein the said carrier comprising at least one water-soluble and/or biodegradable polymer has a concentration ranging from 55-95%.

5. The claim as claimed in claim 1, wherein at least one water-soluble and/or biodegradable polymer is polyvinyl alcohol (PVA), xanthan gum, starch, or combinations thereof.

6. The composition as claimed in claim 1, wherein said composition is used as a biofertilizer, biocontrol agent or combinations thereof.

7. The composition as claimed in claim 1, wherein the composition is lightweight, condensed, has high water solubility and adhesive property.

8. The claim as claimed in claim 6, wherein the biofertilizers and biocontrol agents are used for seed coating, chemical-nutrient coating, soil application, hydroponic application, foliar spray, and/or combinations thereof.

9. The claim as claimed in claim 6, wherein the use of the biofertilizers and biocontrol agents lead to enhanced crop yield, plant growth, root growth, seed germination, crop quality, soil fertility.

10. A process for the preparation of a gel-based agrobiological composition, the said process comprising the steps of:
preparing a solution of a carrier comprising at least one water-soluble and/or biodegradable polymer;
mixing the solution with a concentrate of one or more microbiological entity/s at a density higher than 1×10¹² cfu/mL; to obtain a homogenous gel-based composition;
wherein the said composition has a shelf life of at least 12 months.

11. The claim as claimed in claim 1, wherein the said microbiological entity is one or more bacterial or fungal cells.

12. The claim as claimed in claim 11, wherein the said bacterial cells are selected from Azospirillum brasilense, A lipoferum, Azotobacter chroococcum, Azotobacter vinelandii, Rhizobium leguminosarum, Pseudomonas fluorescens, Bacillus megaterium, Bacillus polymyxa, B. subtilis, Frateuria aurantia, Bacillus mucilaginous, Thiobacillus ferrooxidans, Bacillus aryabhattai, B. thuringiensis, Gluconacetobacter diazotrophicus Pseudomonas striata, P. fluorescence, P. putida, Serratia liquefaciens, S. marcescens, Thiobacillus thioparus, T. neapolitanus, T. denitrificans, T. thiooxidans T. ferrooxidans, T. acidophilus, Paracoccus denitrificans, P. versutus, Xanthobacter tagetidis or a combination thereof.

13. The claim as claimed in claim 11, wherein the said fungal cells are selected from Aspergillus, Fusarium, Penicillium, Piriformospora, Phoma, Funneliformis, Rhizophagus, Glomus, Trichoderma harzianum, T. viride, Metarhizium anisoplae, M. album, M. flavoviride, Beauveria bassiana, Verticillium lecanii, Paecilomyces lilacinus, Ampelomyces quisqualis, Hirsutella thompsonii or a combination thereof.

14. The claim as claimed in claim 11, wherein the concentration of the said microbiological entity is in the range of 1-30% (w/v).

15. The process as claimed in claim 10, wherein the carrier is prepared at a temperature ranging from 50-120° C. and at a pH ranging from of 5.5-7.5.

16. The process as claimed in claim 10, wherein the concentrate of microbiological entity/s and carrier solution are mixed in a ratio ranging from 1:1 to 1:4.

17. A gel-based agrobiological composition comprising:
a carrier comprising at least one of PVA, Xanthan gum, starch, or a combination thereof; and
at least one microbiological entity selected from Azospirillum brasilense, A lipoferum, Azotobacter chroococcum, Azotobacter vinelandii, Rhizobium leguminosarum, Pseudomonas fluorescens, Bacillus megaterium, Bacillus polymyxa, Frateuria aurantia, Bacillus mucilaginous, Thiobacillus ferrooxidans, Bacillus aryabhattai, B. thuringiensis, Gluconacetobacter diazotrophicus Pseudomonas striata, P. fluorescence, Serratia liquefaciens, S. marcescens, Thiobacillus thioparus, T. neapolitanus, T. denitrificans, T. thiooxidans T. ferrooxidans, T. acidophilus, Paracoccus denitrificans, P. versutus, Xanthobacter tagetidis, Aspergillus, Fusarium, Penicillium, Piriformospora, Phoma, Funneliformis, Rhizophagus, Glomus, Trichoderma harzianum, T. viride, Metarhizium anisoplae, M. album, M. flavoviride, Beauveria bassiana, Verticillium lecanii, Paecilomyces lilacinus, Ampelomyces quisqualis, Hirsutella thompsonii or combinations thereof; wherein the said carrier is loaded with the microbiological entity/s at a density higher than 1×10¹² cfu/mL; wherein the said composition has a shelf life of at least 12 months.

18. The claim as claimed in claim 17, wherein the carrier loaded with microbiological entity/s is stable at temperatures ranging from 1°° C. to 60°° C.
